(19) United States
(12) Patent Application Publication
(10) Pub. No.: US 2011/0008394 A1
(43) Pub. Date: Jan. 13, 2011

(54) ACTIVE SUBSTANCES THAT INCREASE THE ACTIVATION THRESHOLD OF IMMUNE CELLS

(75) Inventors: Nicolas Bechetoille, Brignais (FR); Valerie Andre, Ampuis (FR)

Correspondence Address: BASF Beauty Care Solutions France;C/O Connolly Bove Lodge & Hutz LLP, 1007 North Orange Street, P.O. Box 2207, Wilmington, DE, 19899, US

(73) Assignee: BASF Beauty Care Solutions France S.A.S., Lyon (FR)

(21) Appl. No.: 12/922,336

(22) PCT Filed: Mar. 16, 2009
(86) PCT No.: PCT/EP2009/053074

§ 371 Date: Sep. 13, 2010

(30) Foreign Application Priority Data
Mar. 14, 2008 (FR) 0851694

(51) Int. Cl. A61K 36/81; A61K 36/18; A61K 36/185; A61K 36/28; A61K 36/47; A61P 17/00; A61P 37/02

(52) U.S. Cl. 424/278.1; 424/725

(57) ABSTRACT

The present invention relates to the use of active substances that increase the activation threshold of immune cell as an active principle for the preparation of a cosmetic or pharmaceutical composition, especially a dermatological composition. The invention also relates to a method for screening such active substances.

The invention relates to the use of active principles for the preparation of cosmetic or pharmaceutical compositions intended for increasing the activation threshold of skin immune cells and/or mucosa immune cells.

The present invention in particular relates to the care and/or treatment of sensitive skin or skin rendered momentarily sensitive, reactive or hyperreactive.

### PRIOR ART

Cutaneous irritation and allergy reactions (delayed contact hypersensitivity or contact allergy or contact eczema) have become a health problem in industrialized countries. The causes are as varied as the number of contact irritants and allergens found, for example, in metal salts, cosmetic and hygiene products, fragrances, drugs, preserving agents, disinfectants, clothing, plants, etc. In this same context, the number of people who declare that they have sensitive or hyperreactive skin has greatly increased in recent years. This number has risen from 30% of the population in the 1980s to about 60% today.

One of the most important causes of skin sensitivity is related to the weakening of the barrier function, caused, inter alia, by a hereditary/acquired deficiency of the intercellular lipids of the stratum corneum. Increased neurosensory activity, which is characterized by changes in the nerve endings of the epidermis, an accumulation of neurotransmitters or a disruption in the transmission of information in the central nervous system, is also a factor causing an increase in skin sensitivity. A third additional cause of skin sensitivity is increased immune sensitivity especially comprising a measurable increase in the density of the epidermal Langerhans cells (LC), and which may, in the most extreme cases, lead to pathologies such as contact urticaria, contact irritative or allergic dermatitis, or atopic dermatitis.

The polymorphism of sensitive skin types is reflected by subjective sensations such as stinging, a sensation of burning, a sensation of itching, tautness and objective skin reactions such as redness, visible squamae and thickening of the skin. These unsightly and/or uncomfortable manifestations, especially irritation, are characteristic of sensitive skin. In the most extreme cases, irritation or even allergic reactions are also described.

In both cosmetics and pharmaceutics, it is known practice to reduce the sensitivity of all skin types, especially by preventing and/or treating the inflammatory or irritant reaction. Specifically, in the case of individuals whose skin is said to be “sensitive”, an exposure, even minor, to aggressive agents or irritant conditions may be reflected by unsightly and/or uncomfortable cutaneous and/or mucosal manifestations that may lead to an inflammation or a substantial irritation reaction that it would be appropriate to avoid. The current treatments for sensitive skin have the object of making the skin more tolerant, i.e. of raising the reactivity threshold of sensitive skin types, which are characterized by a lower irritability threshold to irritant reactions. These treatments are based on the use of products that “calm” the skin.

Special “sensitive-skin” cosmetic formulations in which the components are often reduced to the minimum and the substances that are, in principle, unsuitable for sensitive or allergic epidermides are excluded therefrom. Mention may be made of the use of moisturizing products containing neither fragrance, nor alcohol, nor dye, nor pigment, nor preserving agents, nor active agents, and having a neutral pH.

Soothing or calmative products based on spring waters, which are rich in trace elements and mineral salts, have properties similar to those of physiological saline solution, such as isotonicity. These products may be enriched with calmative and anti-inflammatory plant extracts such as mallow, oat and cornflower.

Protective products that have the capacity to reinforce the skin barrier with natural molecules which will reconstruct the hydrolipid film of the epidermis in order for the said epidermis to restore all its protective functions. Ceramides, which are precursors of skin lipids, are typically used for this type of application. Products based on saccharides having a film-forming effect are also described for caring for hyperreactive skin.

Destressing products that have neurocosmetic activity, such as β-endorphin-like products, by acting on the sensitive nerve fibre receptors of the skin. These products are generally coupled to anti-inflammatory effects, either by releasing an anti-inflammatory neuromediator such as α-MSH (melanocyte-stimulating hormone), or by inhibiting endogenous anti-inflammatory factors such as TNFα (tumor necrosis factor alpha), IL-1β (interleukin 1 beta) or PGE2 (prostaglandin E2).

The cutaneous immunological component, which fundamentally involves all skin cells, is constituted mainly by:
the cutaneous dendritic cells (DC): the Langerhans cells and the dermal DCs (DDC), which, by virtue of their function as antigen-presenting cells (AgPC), are the cause of the immune responses of the skin;
the keratinocytes, which, by virtue of the fundamental cutaneous microenvironment that they constitute (sweat, sebum, antimicrobial peptides, growth factors, cytokines, chemokines, etc.), are the key cellular partner of the CDs of the skin.

During stimulation by an antigen, for example allergens, pathogens and/or foreign bodies, the skin DCs have the function of:
picking up or capturing the antigen;
migrating outside the skin and in the direction of the lymphatic ganglions;
presenting the antigenic information to the lymphocytes in order to initiate a specific adaptative immunity.

The skin DCs must also integrate all the endogenous signals delivered by the neighbouring keratinocytes. Specifically, under any stimulation and depending on its nature, the keratinocytes produce a very broad panoply of soluble mediators (such as cytokines, etc.) which will influence the immunological functions of the LCs and of the DDCs.

Following capture of the antigen, the skin DCs subsequently undergo phenotypic and functional changes:
capture of the antigen induces the activation of the LCs and DDCs, which then acquire an “activated” phenotype. This activated phenotype is reflected by a strong expression of the co-stimulation molecules CD80 and CD86 that are directly involved in the activation threshold of the T lymphocytes by the DCs. The membrane expression of the molecules of the class II MHC (major histocompatibility complex), and especially HLA-DR, is also greatly increased by virtue of the presentation of the antigenic peptides at the surface of the DCs.
Activation of the LCs and DDCs is also correlated to the acquisition of expression of the CCR7 receptor that is essential for the migration of the DCs outside the skin and in the direction of the lymphatic ganglions.
During their migration, the LCs and the DDCs undergo a maturation process and acquire a “mature” DC phenotype. This mature state, which is essential for sensitization of the lymphocytes and the establishment of an immunity, is reflected by the expression of the markers CD83 and DC-LAMP and by the capacity to secrete cytokines.

### AIM OF THE INVENTION

The main aim of the invention is to provide active principles that are capable of increasing the activation threshold of the immune cells of an individual, in particular the cutaneous and/or mucosal immune cells. This results in a decrease in the cutaneous and/or mucosal sensitivity and/or in the cutaneous and/or mucosal reactivity and/or an increase in the skin and/or mucosal tolerance. This is therefore of undeniable interest in the context of cosmetic or pharmaceutical, in particular dermatological, treatment and/or care.

The aim of the present invention is also to provide a screening model that takes into account the response of the two populations of cutaneous DC (LC and DDC), for screening substances having the activity mentioned above.

### DETAILED DESCRIPTION OF THE INVENTION

The present invention relates to the use of at least one active principle chosen from a plant extract of Phoradendron piperoides, a plant extract of Cestrum latifolium, a plant extract of Spilanthes oleracea, a plant extract of Maprounea guyanensis, securinine, foliosidine acetonide, 8-oxopseudopalmatine, or any combination thereof, as an active agent for increasing the activation threshold of the immune cells of an individual, in a cosmetic composition, or for the preparation of a pharmaceutical composition, and in particular of a dermatological composition, preferably intended for increasing the activation threshold of the immune response of an individual. Such an active principle advantageously makes it possible to reduce the sensitivity and/or reactivity of at least part of the skin and/or mucosa of an individual.

By the term “reduce”, the inventors mean at least partially decrease and/or limit the reactivity of at least part of the skin and/or mucosa of an individual.

Advantageously, the active principle reduces and/or prevents the cutaneous and/or mucosal immune reaction.

Advantageously, the active principle reduces and/or inhibits the activity of at least one type of immune cell, in particular dendritic cells.

Advantageously, the active principle reduces the activity of at least epidermal Langerhans cells (LC) and/or dermal dendritic cells (DDC).

Advantageously, the active principle is in combination with a calmative.

The active principles according to the invention have the capacity to reduce the immune response by increasing the activation threshold of the immune cells, and especially the DCs. Specifically, the active principles according to the invention are advantageously immunorepressive on the immune capacities of the skin, which is fundamentally different from the inflammatory pathway of the skin. The current anti-inflammatory products essentially reduce the capacities for synthesis of the soluble pro-inflammatory factors (cytokines, interleukins, etc.) secreted by the keratinocytes of the skin. The substances according to the invention target an entirely different regulation pathway, which is that of the immune response of the skin. These modes of action may be complementary.

The activation threshold of the immune cells of an individual, in particular of the DCs, is defined by the expression of the activation markers and especially the activation marker CD86. In normal skin, the DCs, which are immature and unactivated, have a basal level of expression of CD86 that we have defined as being the activation threshold of these cells. Thus, reduction of expression of the activation markers, in particular of CD86, on the immature and unactivated DCs is reflected by an increase in the activation threshold of the immune cells.

The invention has the advantage of targeting the DCs of the skin, i.e. of targeting the immune response, in contrast with standard anti-inflammatory products that act solely on indirect pathways of the cutaneous immune response and especially on the secretion of soluble immunity mediators (cytokines) by the cutaneous environment, and especially by the keratinocytes.

Advantageously, the active principle according to the invention reduces in the skin the expression of at least one type of marker chosen from (i) CD86 expressed at a basal level by the immature dendritic cells; (ii) CD40, CD54, CD80, HLA-DR, CCR7 or CD86 expressed by activated dendritic cells; and (iii) CD40, CD54, CD80, HLA-DR, CCR7, CD86, CD83 or DC-LAMP expressed by activated and mature dendritic cells, especially to reduce the reactivity of sensitive skin.

The invention has the advantage of increasing the threshold of triggering of the immune response, while at the same time having a minimal effect on a subsequent immune response when the individual has need thereof, for instance during a bacterial attack of the skin, or in general in the presence of a pathogen agent, a highly aggressive chemical agent and/or foreign element. On the other hand, the anti-inflammatory products that reduce the synthesis of the soluble pro-inflammatory mediators in the skin do not take into account the subsequent response of the DCs of the skin. Thus, in standard treatments with anti-inflammatory products, the immune response is blocked or at the very least impaired. This obviously has the drawback of affording poor protection of the skin of the individual undergoing an attack by an immune response activator.

Thus, advantageously, the active principles according to the invention do not substantially inhibit the immune response, and especially the immunological functionalities of dendritic cells.

Moreover, the active principles according to the invention allow the partial recovery of the basal immune activity, and especially that of the dendritic cells, when the active principle is no longer in contact with the skin after a topical application.

The active principles according to the invention are suitable for any skin type and particularly for cosmetic care and/or cosmetic treatment for individuals with sensitive, reactive and/or hyperreactive skin and/or skin made momentarily sensitive especially by aggressive dermatological treatments such as treatments with vitamin A acid and/or aggressive agents and/or aggressive conditions.

In general, sensitive skin may be defined as skin that no longer tolerates or very barely tolerates aggressive agents, especially environmental agents such as pollutants, climatic factors (wind, cold, heat), exposure to UV, emotional factors, especially stress and/or chemical agents (heavy metals, detergents, compounds contained in cosmetic treatments such as fragrances, preserving agents, alcohols, pH or AHAs, or dermatological treatments such as vitamin A acid) and/or aggressive conditions, especially perspiration, and mechanical aggression such as epilation, shaving and friction. Sensitive skin or skin made momentarily sensitive is not skin of pathological character. It may, nevertheless, react to aggressive agents and/or conditions via unaesthetic and/or uncomfortable cutaneous and/or mucosal manifestations such as stinging, a sensation of burning, a sensation of itching, tautness, redness, visible squamae or thickening of the skin. Thus, the “sensitive skin” character may be estimated by the individual himself with subjective cutaneous sensations or by the dermatologist with objective cutaneous reactions.

The present invention thus allows control of the level of activation and/or maturation of the DCs of the skin to prevent and/or reduce the increased and/or excessive response of sensitive skin, or of skin made momentarily sensitive, reactive or hyperreactive.

According to one implementation variant, the invention covers the cosmetic use of the active principles for preventing and/or treating the unaesthetic and/or uncomfortable cutaneous and/or mucosal manifestations. The active principles according to the invention are thus particularly suitable for cosmetic care and/or cosmetic treatment. They especially allow the preparation of cosmetic compositions with a calmative and/or anti-irritant and/or protective effect in particular against aggressive agents and/or aggressive conditions.

Thus, the invention relates to two different types of care and/or treatment depending on the healthy state (cosmetic care or treatment) or pathological state (dermatological or pharmaceutical care or treatment) of the skin and/or mucosas of the individual concerned.

According to another implementation variant, the invention covers active principles for the preparation of a pharmaceutical composition, in particular a dermatological composition, for preventing and/or treating pathologies caused by activation of the immune response.

A subject of the invention is also a cosmetic composition comprising an active principle chosen from a plant extract of Phoradendron piperoides, a plant extract of Cestrum latifolium or a plant extract of Spilanthes oleracea at a concentration of between 0.001% and 10% by weight, securinine at a concentration of 1×10⁻⁷% to 1%, foliosidine acetonide, 8-oxopseudopalmatine and any combination thereof in a cosmetic vehicle suitable for topical application.

The invention also relates to a pharmaceutical and/or dermatological composition intended for increasing the activation threshold of the immune response, especially of the dendritic cells, especially for preventing and/or treating the reactivity of the skin and/or of the mucosas, comprising an active principle chosen from a plant extract of Phoradendron piperoides, a plant extract of Cestrum latifolium, a plant extract of Spilanthes oleracea, securinine, foliosidine acetonide, 8-oxopseudopalmatine, and any combination thereof, in a pharmaceutical and/or dermatological vehicle suitable for topical application.

The invention also relates to the use of at least one active principle for the preparation of a pharmaceutical composition, and especially a dermatological composition, intended for preventing and/or treating an irritation or cutaneous and/or mucosal allergic reaction, especially a contact reaction, a delayed contact hypersensitivity, a contact eczema, an urticaria, especially contact urticaria, a contact irritative or allergic dermatitis, and/or an atopic dermatitis.

Advantageously, at least one active principle of the invention is used for the preparation of a pharmaceutical composition also comprising an anti-inflammatory agent.

According to one preferential mode, the pathologies are irritation or cutaneous and/or mucosal allergic reaction, especially contact reaction, a delayed contact hypersensitivity, a contact eczema, an urticaria, especially contact urticaria, an irritative or allergic contact dermatitis, and/or an atopic dermatitis. The urticaria is especially a contact urticaria and/or a dietary urticaria causing cutaneous and/or mucosal manifestations.

Preferably, the pharmaceutical and/or dermatological composition is intended for preventing and/or treating an irritation or cutaneous and/or mucosal allergic reaction, especially a contact reaction, a delayed contact hypersensitivity, a contact eczema, an urticaria, especially a contact urticaria, an irritative or allergic contact dermatitis, and/or an atopic dermatitis.

Activation of the immune response of the skin and/or of the mucosas is characterized by the activation of at least one type of immune cell, and especially the dendritic cells (DC), for instance the epidermal Langerhans cells (LC) and/or the dermal dendritic cells (DDC).

The DCs of the skin are characterized by their specific markers, the LCs and the DDCs specifically expressing langerine and DC-SIGN, respectively.

Immature dendritic cells are defined by a basal phenotypic state before activation with activating and/or maturation agents, and especially of TNFα and/or LPS (lipopolysaccharide) type.

“Activated” dendritic cells are defined by an activated phenotypic state comparable to that induced with activating and/or maturation agents, especially of the TNFα and/or LPS type.

An activating and/or maturation agent, i.e. an agent that increases the expression of at least one DC marker, is a danger signal for the DCs, which triggers an immune response. This agent is not limited. These agents are in particular chemical or biological agents, for instance LPS or TNFα, a parameter that irritates the skin such as a stream of gas, a pollutant, a temperature below 15° C. or above 40° C., used or applied to increase the expression of at least one DC marker.

The invention in particular relates to mammals, and in particular to man.

The active principles according to the invention are plant extracts or characterized molecules. The extracts are advantageously derived from a plant chosen from the group consisting of: Phoradendron piperoides, Cestrum latifolium, Spilanthes oleracea, Maprounea guyanensis and any mixture thereof. The preferred characterized molecules are chosen from the group consisting of: securinine, foliosidine acetonide and 8-oxopseudopalmatine. A mixture of two or more of the abovementioned active principles is also covered by the present invention, in any proportion thereof. According to one particularly advantageous mode, the active principle is a plant extract of Cestrum latifolium and/or securinine.

The plant extracts that are useful according to the invention are obtained according to the standard methods in the field.

Depending on the plants, it is advantageous to extract, for example by maceration, at least part of the plant chosen from a root, a rhizome, a stalk, a bark, a flower, a fruit, a sprout, a seed and a leaf, preferably between 1% and 10% (w/w) in a solvent or a mixture of solvents, preferably a polar protic solvent, and advantageously in water, an alcohol, a glycol, a polyol, a water/alcohol, water/glycol or water/polyol mixture (such as water mixed with ethanol, glycerol, butylene glycol or other glycols, such as xylitol, etc.) of from 100/0 to 0/100 (v/v). The extracts obtained are then preferably centrifuged and/or filtered and/or distilled in order to recover the active soluble fraction (crude extract).

The plant extract is preferentially dissolved in a solvent, such as water, an alcohol, a polyol, a glycol, or a mixture thereof. According to the invention, the plant extract is preferentially used at a concentration of between 0.001% and 10% by weight, advantageously between 0.01% and 5% and more particularly at 1% by weight relative to the weight of the composition.

The active substance may be concentrated by evaporating off the solvent, for example by freeze-drying or by atomization.

The characterized molecules (active principles) are advantageously prepared by dissolution in a polar solvent, for instance water, ethanol, glycol or DMSO.

According to the invention, the characterized molecules are preferably used at a concentration of between 1×10⁻⁷% and 1% by weight, advantageously between 1×10⁻⁵% and 1×10⁻¹% and in particular at 1×10⁻¹% by weight relative to the total weight of the composition.

Preferably, the composition is a cosmetic or pharmaceutical composition, especially a dermatological composition, preferably applied topically.

For example, at a rate of 0.5% to 10% of active principle in the final cosmetic formulation and at an application rate of 1 mg of finished cosmetic product per cm² of skin, the amounts of active principles applied per cm² of skin are advantageously between 10⁻¹³ g and 10⁻¹² g for the characterized molecules and between 10⁻⁵ g and 10⁻⁴ g for the active principles of plant origin (plant extract of between 0.001% and 10% by weight in a solvent).

The compounds according to the present invention are preferentially used in the form of cosmetic or pharmaceutical compositions, and preferentially dermatological compositions.

The composition may contain any suitable solvent and/or any suitable vehicle and/or any suitable excipient, optionally in combination with other compounds of interest.

As a result, for these compositions, the excipient contains, for example, at least one compound chosen from the group consisting of preserving agents, emollients, emulsifiers, surfactants, moisturizers, thickeners, conditioning agents, mattifying agents, stabilizers, antioxidants, texturing agents, sheen agents, film-forming agents, solubilizing agents, pigments, dyes, fragrances and sunscreens. These excipients are preferably chosen from the group consisting of amino acids and derivatives thereof, polyglycerols, esters, cellulose polymers and derivatives, lanolin derivatives, phospholipids, lactoferrins, lactoperoxidases, sucrose-based stabilizers. E vitamins and derivatives thereof, natural and synthetic waxes, plant oils, triglycerides, unsaponifiable materials, phytosterols, plant esters, silicones and derivatives thereof, protein hydrolysates, jojoba oil and derivatives thereof, lipo/water-soluble esters, betaines, amine oxides, plant extracts, sucrose esters, titanium dioxides, glycines and parabens, and more preferably from the group consisting of butylene glycol, steareth-2, steareth-21, glycol-15 stearyl ether, cetearyl alcohol, phenoxyethanol, methylparaben, ethylparaben, propylparaben, butylparaben, butylene glycol, natural tocopherols, glycerin, dihydroxycetyl sodium phosphate, isopropyl hydroxycetyl ether, glycol stearate, triisononanoin, octyl cocoate, polyacrylamide, isoparaff in, laureth-7, a carbomer, propylene glycol, glycerol, bisabolol, a dimethicone, sodium hydroxide. PEG-30 dipolyhydroxystearate, capric/caprylic triglycerides, cetearyl octanoate, dibutyl adipate, grapeseed oil, jojoba oil, magnesium sulfate. EDTA, a cyclomethicone, xanthan gum, citric acid, sodium lauryl sulfate, mineral waxes and oils, isostearyl isostearate, propylene glycol dipelargonate, propylene glycol isostearate. PEG-8, beeswax, hydrogenated palm kernel oil glycerides, hydrogenated palm oil glycerides, lanolin oil, sesame oil, cetyl lactate, lanolin alcohol, castor oil, titanium dioxide, lactose, saccharose, low-density polyethylene and an isotonic saline solution.

Advantageously, the abovementioned compositions are formulated in a form chosen from the group consisting of an aqueous or oily solution, an aqueous gel or cream or an oily gel, especially in a jar or a tube, especially a shower gel or a shampoo; a milk; an emulsion, a microemulsion or a nanoemulsion, especially oil-in-water or water-in-oil or multiple or silicone-based; a lotion, especially in a glass, plastic or dose-metering bottle or as an aerosol; an ampoule; a liquid soap; a dermatological bar; an ointment; a mousse; an anhydrous product, which is preferably liquid, pasty or solid, for example in the form of a stick, especially in the form of a lipstick or of tablets; gel capsules; syrups; granules; powders.

The term “topical application” used herein means the application of the composition according to the invention to the surface of the skin and/or mucosa, especially by direct application or by vaporization.

The invention also covers an oral administration, and especially nutraceutical applications.

The term “suitable cosmetic or dermatological vehicle” used herein means that the composition or the components thereof are suitable for use on contact with human skin without any undue toxicity, incompatibility, instability, allergic response, or equivalents thereof.

Many cosmetically active ingredients are known to those skilled in the art for improving the health and/or physical appearance of the skin. A person skilled in the art knows how to formulate cosmetic and/or dermatological compositions to obtain the best effects. Moreover, the compounds described in the present invention may have a synergistic effect when they are combined with each other. These combinations are also covered by the present invention. The CTFA Cosmetic Ingredient Handbook, Second Edition (1992) describes various cosmetic and pharmaceutical ingredients commonly used in the cosmetic and pharmaceutical industry, which are in particular suitable for topical use. Examples of these classes of ingredients comprise, without being limited thereto, the following compounds: abrasive agents, absorbents, compounds for aesthetic purposes, such as fragrances, pigments, dyes, essential oils, astringents, etc. (for example: clove oil, menthol, camphor, eucalyptus oil, eugenol, menthyl lactate, hamelis distillate), antiacne agents, antiflocculants, antifoams, antimicrobial agents (for example: iodopropyl butylcarbamate), antioxidants, binders, biological additives, buffers, swelling agents, chelating agents, additives, biocidal agents, denaturing agents, thickeners, and vitamins, and derivatives or equivalents thereof, film-forming materials, polymers, opacifiers, pH adjusters, reducing agents, depigmenting agents or lightening agents (for example: hydroquinone, kojic acid, ascorbic acid, magnesium ascorbyl phosphate or ascorbyl glucosamine), and conditioning agents (for example: humectants).

In particular, to improve the state of sensitivity of the skin as fully as possible, it is particularly advantageous according to the invention to combine the active principles according to the invention with one or more other active agents chosen from the characterized molecules and plant extracts known for their properties:
neurocosmetics,
external analgesics,
skin calming agent, especially. PLA2 inhibitors, exopolysaccharides of bacterial origin, especially cosmetic calming agent such as the extract of Aesculus hippocastanum (CAS 8053-39-2), the extract of Alteromonas ferment described in patent EP 0 987 010, active components described in patent FR2847267 and notably an extract of Pueraria lobata roots and marketed by Applicant under name Inhipase™.
wound healing agent such as panthenol and derivatives thereof, for example ethyl panthenol. Aloe vera, pantothenic acid and derivatives thereof, allantoin, bisabolol, and dipotassium glycyrrhizinate,
anti-inflammatory agents such as steroidal and non-steroidal anti-inflammatory drugs, in particular inhibitors of production of cytokines and chemokines, of cyclooxygenase, of nitric oxide (NO) and NOS nitric oxide synthase. Examples of anti-inflammatory products that may be mentioned include extracts of Gingko biloba, trilactone terpenes such as gingkolides, especially gingkolide B and bilobalide known for their an agonist properties on platelet activation factor (PAF).
elastase inhibitor such as Haslea ostrearia extract for instance Blue algae extract™ marketed by Applicant
moisturising and/or anti-inflammatory agent such as Palmaria palmate extract for instance Sea Parsley™ product marketed by the Applicant.

The invention also relates to a cosmetic composition comprising an active principle according to the invention in combination with other active principles known for the cosmetic care and/or treatment of sensitive skin or of skin made momentarily sensitive, reactive and/or hyperreactive.

The invention covers a therapeutic treatment method comprising the administration of an active agent for the various treatments listed above to an individual in need thereof, especially via topical application.

The present invention also relates to a method for screening these active principles.

According to one variant, the invention relates to a method for screening active principles capable of increasing the activation threshold of the immune response of cutaneous and/or mucosal dendritic cells, the said method comprising:

(i) the preparation of a culture medium comprising immature or activated dendritic cells (DCs);

(ii) the placing in contact of the medium comprising the DCs with at least one substance to be screened as regards its capacity to increase the activation threshold of the immune response of cutaneous dendritic cells;

(iii) the recovery of the DCs to quantify the expression of the DC activation markers;

(iv) the comparison of the value of the expression of the activation markers of DCs that have been placed in contact with substances to be screened, relative to the value of the DC activation marker expression obtained with a positive control such as a steroidal anti-inflammatory agent, preferentially dexamethasone, to select active principles that increase the activation threshold of the immune response of cutaneous and/or mucosal dendritic cells.

According to another variant, the invention relates to a method for screening active principles capable of increasing the activation threshold of the immune response of cutaneous and/or mucosal dendritic cells, the said method comprising:

(i) the preparation of a culture medium comprising immature or activated dendritic cells (DCs);

(ii) the placing in contact of the medium comprising the DCs with at least one substance to be screened as regards its capacity to increase the activation threshold of the immune response of cutaneous dendritic cells and with at least one agent known for activating the immune response by increasing the expression of at least one DC marker;

(iii) the recovery of the DCs to quantify the expression of the DC activation markers after treatment with a substance to be screened or an active principle and stimulation with the said agent;

(iv) the comparison of the value of the expression of the activation markers of DCs that have been placed in contact with the substance to be screened and the said agent, relative to the value of the DC activation marker expression obtained with a positive control such as a steroidal anti-inflammatory agent, preferentially dexamethasone, to select active principles that increase the activation threshold of the immune response of cutaneous and/or mucosal dendritic cells in the presence of the said agent.

The agent for increasing the expression of at least one DC marker may be an agent known for stimulating the response of immune cells, such as a mixture of LPS and TNFα.

Advantageously, the method also comprises:

(iiib) the reculturing of some of the DCs recovered in step (iii), the said culture medium not comprising any active principle or substance to be screened, for a time that is sufficient for these cells to recover their basal activation level;

(ivb) the quantification of the expression of the DC activation markers following step (iiib) to select the active principles that increase the activation threshold of the immune response of the cutaneous dendritic cells and that allow activation of the immune response following step (iiib). A substance to be screened as regards its capacity to increase the activation threshold of the immune response of cutaneous dendritic cells is a substance such as, for example, a substance of plant origin or a characterized molecule whose capacity for repressing the expression of a DC activation marker it is desired to quantify.

A characterized molecule is a molecule whose chemical structure is known.

The DC activation markers are preferably chosen from: CD40, CD54, CD80, CD86, HLA-DR, CCR7, which is the MIP-3β (macrophage inflammatory protein-3 beta) chemokine receptor.

The DC maturation markers are preferably chosen from: CD83 and DC-LAMP.

To quantify the expression of the DC activation markers, any method may be used. A method of RT-PCR, especially quantitative, or of flow cytometry or of ELISA, may advantageously be used.

The expression of the DC activation markers is thus directed both towards the protein expression of the DC activation markers and the gene expression (mRNA) of the DC activation markers.

The comparison of the value of the expression of the DC activation markers for selecting the active principles relative to the control is performed with positive and negative controls, which are preferably activating and/or maturation agents, for example an LPS/TNFα mixture, and immunosuppressants, for example dexamethasone.

Advantageously, the screened substance is considered as being an active principle or active substance when it makes it possible to obtain according to the screening method a protein expression of CD86 expressed by the LCs or DDCs of less than or equal to 95%, and preferably less than 70%, of the protein expression of CD86 expressed by LCs or DDCs in the presence or absence of an activating agent constituted by LPS 50 μg/mL and TNFα 10 ng/mL, and/or a gene expression of CD86 expressed by LCs or DDCs of less than or equal to 95%, preferably less than 75%, of the gene expression of CD86 expressed by LCs or DDCs in the presence or absence of an activating agent constituted by LPS 50 μg/mL and TNFα 10 ng/mL.

According to other embodiment, the present invention relates to the topical use, preferably non-therapeutic use, of active principles according to the invention in combination with irritant agent or potential irritant agent.

The present invention also relates to composition comprising actives principles according to the invention and irritant agent intended to be in contact with skin and/or mucousa. Irritant agents may be for instance detergents, contact aggressive agents, preserving agent in particular glycol derivatives notably pentylen glycol derivatives, cosmetic irritating agents such as AHA specially, glycolic acid, lactic acid and mixture thereof, such as retinol and retinoid derivatives, specially retinaldehyde, retinoic acid, such as salicylic acid, and/or in combination with one or several components listed in EEC Directive 76/768 allowed under restricted conditions in cosmetic compositions, specially components listed in Annex 3.

According to other embodiment, actives principles according to the invention can be used in daily cosmetic care compositions. The present invention thus concerns cosmetic care and/or treatment method in which at least one active principle according to the invention, preferably Cestrum latifolium extract is daily applied at efficacy dosage on skin and/or mucosa, possibly in combination with cosmetic irritating agents.

Other aims, characteristics and advantages of the invention will emerge clearly to a person skilled in the art on reading the explicative description, which makes reference to examples that are given for purely illustrative purposes and that should not in any way be considered as limiting the scope of the invention.

The examples form an integral part of the present invention and any characteristic appearing to be novel relative to any prior art from the description taken in its entirety, including the examples, forms an integral part of the invention in its function and in its generality.

Thus, each example has a general scope.

Moreover, in the examples, all the percentages are given on a weight basis, unless otherwise indicated, the temperature is expressed in degrees Celsius, unless otherwise indicated, and the pressure is atmospheric pressure, unless otherwise indicated.

### Examples

### Example 1

### Preparation of Skin Dendritic Cells, LCs and DDCs

The process for preparing LCs and DDCs from blood monocytes is performed in two successive steps.

Step 1: Process for Separating Monocytes from Circulating Peripheral Blood

The circulating venous peripheral blood of one or more human donors is collected in the presence of anticoagulants, preferably lithium heparin.

Separation of the monocytes from the circulating blood may be performed advantageously according to the following protocols:

1/ After centrifuging the blood on a lymphocyte separation medium, the mononuclear cells are recovered and then:
Either marked with an antibody cocktail, for instance anti-CD3, anti-CD7, anti-CD16, anti-CD19, anti-CD56, anti-CD123, anti-glycophorin A antibodies coupled to magnetic beads. After passing through a magnetized column, only the unmarked monocytes are eluted and recovered.
Or marked with an antibody specific for monocytes, such as an anti-CD14 antibody coupled to magnetic beads. After passing through a magnetic column, only the marked monocytes are retained in the column. After eluting the column, the marked monocytes are recovered.
Or marked with an antibody specific for monocytes, such as an anti-CD16 antibody coupled to a fluorochrome, such as phycoerythrin. After cell sorting by flow cytometry, only the marked monocytes are recovered.

2/ The monocytes are recovered by performing any physical separation process well known to those skilled in the art and especially by sedimentation or centrifugation, and are eluted without further processing for the subsequent culturing.

3/ Per 100 mL of collected blood, the extraction yield is about 150 million (±20 million) mononuclear cells, out of which up to 40 million monocytes are obtained.

Step 2: Process for Freezing the Monocytes from Circulating Peripheral Blood

The monocytes, as obtained in step 1, may be advantageously frozen from −80° C. to −196° C., and preferably cryofrozen at −196° C. in liquid nitrogen, at a rate of about 10 million per mL, in a suitable freezing medium, for instance a medium composed of 90% foetal calf serum and 10% DMSO (dimethyl sulfoxide).

This freezing step advantageously allows the preparation of a donor pool, for instance the cellular mixture of 3 monocyte donors.

Step 3: Process for Culturing the Isolated Monocytes to Obtain Immature LCs and DDCs

The monocytes, as obtained in steps 1 and/or 2, are cultured (37° C., 5% CO₂) at a rate of about 1 million per mL, in a defined or undefined culture medium, preferably in RPMI 1640 medium supplemented with 10% foetal calf serum, and containing the following three cytokines: GM-CSF (granulocyte-macrophage colony-stimulating factor, at a rate of 200 ng/mL), TGFβ1 (transforming growth factor β1). at a rate of 10 ng/mL, and IL-13 (interleukin 13) at a rate of 10 ng/mL.

On the 6th day of culturing, immature LCs and DDCs are simultaneously generated and are phenotypically and functionally very similar to their in vivo homologues:
up to 40% of the dendritic cells generated in vitro express langerine intracellularly and up to 60% express DC-SIGN,
the LCs and DDCs generated in vitro are immature since they express at a basal level the co-stimulation molecules CD80 and CD86, and do not express the maturation molecules CD83 and DC-LAMP,
after stimulation with TNFα (advantageously 10 ng/mL) and LPS (advantageously 50 μg/mL) for 48 hours, the LCs and DDCs acquire a phenotype of activated and mature DCs as demonstrated by an increase in the expression of the molecules CD80 and CD86 and by the acquisition of expression of the maturation markers CD83 and DC-LAMP.

Step 4: Process for Culturing the Isolated Monocytes to Obtain Activated LCs and DDCs

The cells are cultured as described in Example 1 and step 3, but in the presence of the following three cytokines: GB-CSF at a rate of 200 ng/mL, TGFβ1 at a rate of 10 ng/mL, and TNFα at a rate of 10 ng/mL.

After incubating for 24 hours, activated LCs and DDCs are simultaneously generated and are phenotypically and functionally very similar to their in vivo homologues:
The LCs and DDCs show strong expression of the following molecules: HLA-DR, which is a class II MHC molecule, CD80 and CD86, which are co-stimulation molecules, and CCR7, which is the migration receptor.
The LCs and DDCs also show high migration capacity in response to the chemokine MIP-3β.

### Example 2

### Preparation of Cosmetic Active Principles

The active principles are of different origins (plant origin or characterized molecules, for example).

Depending on the plant, it is advantageous to extract, for example by maceration, at least part of the plant chosen from a root, a rhizome, a stalk, a bark, a flower, a fruit, a sprout, a seed and a leaf, preferably between 1% and 10% (w/w) in a solvent or a mixture of solvents, preferably a polar protic solvent, and advantageously in water, an alcohol, a glycol, a polyol, a water/alcohol, water/glycol or water/polyol mixture (such as water mixed with ethanol, glycerol, butylene glycol or other glycols, such as xylitol, etc.) of from 100/0 to 0/100 (v/v). The extracts obtained are then preferably centrifuged and/or filtered and/or distilled in order to recover the active soluble fraction (crude extract). The active substance is advantageously the plant extract in a solvent, such as water, an alcohol, a polyol, a glycol, or a mixture thereof, and preferably in water. The crude extract dissolved in the solvent (active principle) is used for the tests preferably at a concentration of between 0.001% and 10% (v/v), advantageously between 0.01% and 5% and more particularly at 1% by weight of the crude extract relative to the total weight of the active principle. The active substance may be concentrated by evaporating off the solvent, for example by freeze-drying or by atomization.

The characterized molecules are prepared by dissolution in a solvent, for instance water, ethanol, glycol or, preferably, in DMSO, and are preferably tested at a concentration of between 1×10⁻⁷% and 1%, advantageously between 1×10⁻⁵% and 1×10⁻¹%, and in particular at 1×10⁻¹% by weight of the active principle.

### Example 3

### Model for Screening Cosmetic Active Principles

The active agents are tested on the LCs/DDCs obtained according to Example 1. When the active principles are tested on the “immature” LCs/DDCs obtained in step 3 of Example 1, this is referred to as preventive care. When the active agents are tested on the “activated” LCs/DDCs obtained in step 4 of Example 1, this is referred to as curative care.

The cells are inoculated into 24-well plates, for example at a rate of 400 000 cells per cm², in a defined or undefined culture medium, preferably in RPMI 1640 medium supplemented with 10% foetal calf serum, and containing the following two cytokines: GM-CSF at a rate of 200 ng/mL and TGFβ1 at a rate of 10 ng/mL. The active principles are then added to the said culture medium and tested at the following final concentrations:
for the plant extracts, between 0.001% and 10% (v/v), advantageously between 0.01% and 5% and in particular at 1% by weight of the active principle relative to the total weight of the culture medium;
for the characterized molecules, between 1×10⁻⁷% and 1% (v/v), advantageously between 1×10⁻⁵% and 1×10⁻¹%, and in particular at 1×10⁻¹% by weight of characterized molecule (or chemical molecule) relative to the total weight of the culture medium.

The active principles are incubated for up to 48 hours and advantageously for 24 hours in the said culture medium at 37° C. under 5% CO₂.

The negative controls are either the culture medium alone, or the culture medium containing the solvent used during the process for extracting the extracts tested at the concentration equivalent to that of the tested active agent, or the culture medium containing dexamethasone (10⁻⁸ M), which is a mainly anti-inflammatory glucocorticoid. The reference positive control used in order to induce an LC/DDC activation is a mixture of LPS (at 50 μg/mL advantageously) and of TNFα (10 ng/mL advantageously).

### Example 4

### Selected Active Principles with Immunosuppressant Capacities

We selected 3 characterized molecules (securinine, foliosidine acetonide and 8-oxopseudopalmatine) and also 4 plant extracts (Cestrum latifolium, Phoradendron piperoides, Maprounea guyanensis and Spilanthes oleracea), which are presented in Tables 1 and 2, respectively.

TABLE 1
Characterized molecules selected in this invention

| CHARACTERIZED | CAS | CHEMICAL | MOLAR |
|---|---|---|---|
| MOLECULES | NUMBER | FORMULA | MASS |
| Securinine | 5610-40-2 | C_{₁₃}H_{₁₅}NO_{₂} | 217.27 g.mol^{^{−}}^{¹} |
| Foliosidine acetonide | nd | C_{₂₁}H_{₂₁}NO_{₅} | 367.40 g.mol^{^{−}}^{¹} |
| 8-Oxopseudopalmatine | 10211-78-6 | C_{₁₉}H_{₂₅}NO_{₅} | 347.41 g.mol^{^{−}}^{¹} |

TABLE 2
Plant extracts selected in this invention

| PLANT | PART OF |
|---|---|
| EXTRACTS | THE PLANT |
| Cestrum latifolium | aerial part |
| Phoradendron piperoides | aerial part |
| Maprounea guyanensis | leaf |
| Spilanthes oleracea | leaf |

### Example 5

### Test of Cytotoxicity of the Cosmetic Active Principles

For each active agent tested, a cell viability test is performed on the cells in order to show that the active agents have no cytotoxic effect on the cells and that the effect of the active agent is not the result of a cellular stress associated with a mortality induced by the active principles. The active principles have a cytotoxic effect when the cell viability is less than 75% after treatment with the said active agents.

The LCs/DDCs, as described in Example 1 and step 3, are inoculated into P96-well plates, at a rate of 200 000 cells/well, and in the culture medium described in Example 3. The treatment of the cells with the active principles is described in Example 3.

After treatment with the said active principles, the cells are incubated with 5 μl of dye 7-AAD (7-aminoactinomycin D) per well. After incubation for 5 minutes, the cells are analysed by flow cytometry.

The cell viability is expressed as a percentage of viable cells or negative 7-AAD, the dye not penetrating the membranes that are impermeable. This analysis technique also makes it possible to visualize the cells positively marked with 7-AAD, i.e. the cells that have undergone a cytotoxic effect via the active agent.

The negative controls are either the culture medium alone, or the culture medium containing the solvent used during the process for extracting the extracts tested at the concentration equivalent to that of the active agents tested. The reference positive control used in order to induce a mortality of the LCs/DDCs is camptothecin at a concentration of 4 μM final.

Table 3 shows the results of the study of cell viability for the active principles selected in this invention.

TABLE 3
Study of the cell viability of immature LCs/DDCs
after treatment with the selected active principles

| ACTIVE | CONCENTRATION | CELL | CYTOTOXIC |
|---|---|---|---|
| PRINCIPLES | 24 hour treatment | VIABILITY | EFFECT |
| Securinine | 10^{^{−}}^{⁷}M | 94% | NO |
| Foliosidine | 10^{^{−}}^{⁷}M | >95% | NO |
| acetonide | | | |
| 8-Oxopseudo- | 10^{^{−}}^{⁷}M | 94% | NO |
| palmatine | | | |
| Cestrum | 1% | 89% | NO |
| latifolium | | | |
| Phoradendron | 0.05% | 93% | NO |
| piperoides | | | |
| Maprounea | 0.05% | >95% | NO |
| guyanensis | | | |
| Spilanthes | 0.05% | 91% | NO |
| oleracea | | | |
| Camptothecin | 3 μm | 35% | YES |

Conclusion:

The cosmetic active principles described in Table 3 show no cytotoxic effect on the LCs/DDCs. The cell viability of the LCs/DDCs after treatment with the said active principles is close to 100%.

### Example 6

### Analysis of the Immunorepressive Properties of the Cosmetic Active Principles—Study of the Protein Expression of the Molecule CD86

Evaluation of the immunosuppressant potential of the active principles requires a study of the expression of the activation molecules on LCs/DDCs treated with the said active principles, for instance the co-stimulation molecule CD86.

Flow cytometry makes it possible to quantify the protein expression of CD86 on the cell membrane of the LCs/DDCs treated or otherwise with the said active agents.

The study of the immunorepressive properties of the cosmetic active principles by flow cytometry proceeds in the following manner:

1/ The immature LCs/DDCs are generated as described in Example 1 and step 3.

2/ The cells are inoculated in P24-well plates, at a rate of 400 000 cells per cm², and in the culture medium described in Example 3. The treatment of the cells with the active principles is described in Example 3. In parallel, the cells are treated with decreasing doses of active principles in order to establish a dose/effect correlation: from 10⁻⁶ M to 5×10⁻⁸ M for the characterized molecules and from 0.1% to 0.005% for the plant extracts.

3/ After treatment with the active agents, the cells are recovered and then inoculated in P96-well plates, at a rate of 200 000 cells/50 μl/well, in a marking medium (30% RPMI, 68% PBS and 2% FCS).

4/ The cells are then incubated for 30 minutes with an anti-CD86 monoclonal antibody coupled to a fluorochrome, such as phycoerythrin (advantageously 10 μl of anti-CD86 antibody/well). The negative marking control corresponds to the IgG1 control isotype. After this marking step, the cells are rinsed with the said marking buffer, and then analysed by flow cytometry.

5/ Table 4 shows the results of the analysis of the immunorepressive properties of the selected active principles. The results are expressed in the following manner: the control not treated with the active agent is referenced as 100% expression of the molecule CD86, and the tests are expressed as the residual expression (in %) of the molecule CD86 after treatment with the active principles. The negative control corresponds to the cells treated with dexamethasone, which has the property of reducing the expression of CD86. The level of immunorepression, which is inversely proportional to the residual expression of CD86, was also calculated.

TABLE 4
Immunorepressive properties of the active principles tested on immature LCs/DDCs

| ACTIVE | CONCENTRATION | RESIDUAL | LEVEL OF |
|---|---|---|---|
| PRINCIPLES | 24 hour treatment | EXPRESSION OF CD86 | IMMUNOREPRESSION |
| 8-Oxopseudopalmatine | 10^{^{−}}^{⁷}M | 81% | 0.14 |
| Securinine | 10^{^{−}}^{⁷}M | 83% | 0.10 |
| Foliosidine acetonide | 10^{^{−}}^{⁷}M | 85% | 0.17 |
| Phoradendron piperoides | 0.05% | 93% | 0.07 |
| Cestrum latifolium | 1% | 51% | 0.49 |
| Spilanthes oleracea | 0.05% | 68% | 0.32 |
| Maprounea guyanensis | 0.05% | 80% | 0.2 |
| Dexamethasone | 10^{^{−}}^{⁸}M | 50% | 0.5 |

Conclusion:

The active principles described in Table 4 have the capacity to reduce the protein expression of the co-stimulation molecule CD86. The said active principles thus have the capacity of increasing the activation threshold of the LCs/DDCs. In other words, the higher and the closer to 1 the level of immunorepression, the greater the immunorepressive power of the active agent.

TABLE 5
Dose/effect study of the active principles tested on the
immature LCs/DDCs

| ACTIVE | RESIDUAL EXPRESSION OF CD86 | | | |
|---|---|---|---|---|
| PRINCIPLES | 10^{^{−}}^{⁶}M | 5 × 1^{⁰}^{^{−}}^{⁷}M | 10^{^{−}}^{⁷}M | 5 × 10^{^{−}}^{⁸}M |
| 8-Oxopseudopalmatine | 85% | 92% | 81% | 100% |
| Securinine | 100% | 100% | 83% | 90% |
| Foliosidine acetonide | 83% | 80% | 85% | 85% |

TABLE 6
Dose/effect study of the active principles tested
on the immature LCs/DDCs

| ACTIVE | RESIDUAL EXPRESSION OF CD86 | | | |
|---|---|---|---|---|
| PRINCIPLES | 1% | 0.50% | 0.10% | 0.05% |
| Phoradendron piperoides | 158% | 154% | 132% | 93% |
| Cestrum latifolium | 51% | 70% | 139% | 127% |
| Spilanthes oleracea | 170% | 122% | 75% | 68% |
| Maprounea guyanensis | 116% | 95% | 92% | 80% |

Conclusion:

The active principles described in Tables 5 and 6 have immunorepressive properties that are dependent on their final concentrations.

### Example 7

### Analysis of the Immunorepressive Properties of the Cosmetic Active Principles—Study of the Expression of Messenger RNAs Coding for the Molecule CD86

Quantitative RT-PCR makes it possible to quantify the expression of the messenger RNAs coding for the protein CD86 in the LCs/DDCs treated or not treated with the said active agents.

The study of the immunorepressive properties of the cosmetic active principles by quantitative RT-PCR proceeds in the following manner:

1/ The immature LCs/DDCs are generated as described in Example 1 and step 3.

2/ The cells are inoculated in P24-well plates, at a rate of 400 000 cells per cm², and in the culture medium described in Example 3. The treatment of the cells with the active principles is described in Example 3.

3/ After treatment with the active principles, the cells are recovered and stored, for example by dry-freezing at −80° C. after rinsing with pH 7.4 phosphate buffer.

4/ The total RNA of the cells is extracted, for example using a 96-well extraction kit on silica columns, and assayed on e96-well spectrophotometer at 260 nm (purity indicator: assay of the proteins at 280 nm). The RNAs are diluted, for example, to 5 ng/μl.

5/ The qualitative RT-PCR in step 1 is performed, for example, on 50 ng of initial RNA on a 96-well plate, on the genes for actin and for the molecule CD86. The specific primers for each gene are specified in Table 7 below and are used, for example, at 0.5 μM.

Specific primers for the genes studied

| | |
|---|---|
| Actin sense sense | |
| (SEQ ID NO 1) | GTGGGGCGCCCCAGGCACCA |
| Actin antisense | |
| (SEQ ID NO 2) | CTCCTTAATGTCACGCACGATTTC |
| CD86 sense | |
| (SEQ ID NO 3) | CTCTTTGTGATGGCCTTCCTGC |
| CD86 antisense | |
| (SEQ ID NO 4) | CCTGTGGGCTTTTTGTGATGGA |

The amplification parameters were advantageously the following: the real-time RT-PCR technique is performed with the Quanti Tect SYBR Green RT-PCR kit (Qiagen, France) on the wells containing the messenger RNA, in an Opticon thermocycler, which performs the amplification cycles. The reverse transcription (RT) is performed for 30 minutes at 50° C., followed by 15 minutes at 95° C. to inhibit the reverse transcriptase, to activate the polymerase and to denature the complementary DNA (cDNA) obtained. 50 cycles of chain polymerization (PCR) are performed (15 seconds at 95° C., 30 seconds at 60° C., 30 seconds at 72° C.). At the end of each cycle, the fluorescence, which is proportional to the number of amplified fragments, is read. The level of expression is defined by the ratio of expression of each gene relative to actin.

6/ Table 8 shows the results of the analysis of the immunorepressive properties of the selected active principles. The results are expressed in the following manner: the untreated control is referenced at 100% expression of the messenger RNAs coding for the molecule CD86, and the tests are expressed as the level of expression of the messenger RNAs coding for CD86 as a percentage of variation relative to those obtained for the negative control.

TABLE 7
Immunorepressive properties of the active principles tested on the immature
LCs/DDCs

| ACTIVE | CONCENTRATION | RESIDUAL | LEVEL OF |
|---|---|---|---|
| PRINCIPLES | 24 hour treatment | EXPRESSIONOF CD86 | IMMUNOREPRESSION |
| Securinine | 10⁻⁷M | 74% | 0.26 |
| Foliosidine acetonide | 10⁻⁷M | 91% | 0.09 |
| 8-Oxopseudopalmatine | 10⁻⁷M | 59% | 0.41 |
| Cestrum latifolium | 1% | 59% | 0.41 |
| Phoradendron piperoides | 0.05% | 82% | 0.18 |
| Maprounea guyanensis | 0.05% | 86% | 0.14 |
| Spilanthes oleracea | 0.05% | 55% | 0.45 |

Conclusion:

The active principles described in Table 7 have the capacity to reduce the expression of the messenger RNAs coding for the co-stimulation molecule CD86. The said active principles thus have the capacity to increase the activation threshold of LCs/DDCs. In other words, the higher and closer to 1 the level of immunorepression, the greater the immunorepressive power of the active agent.

### Example 8

### Study of the Capacities for Response to a Danger Signal of the LCs/DDCs Treated with the Immunorepressive Active Principles

Evaluation of the immunorepressive potential of the active principles also requires a study of the capacity of the LCs/DDCs to respond to a danger signal, for example a bacterial infection, when they are treated with the said active principles. Specifically, we checked that the LCs/DDCs that are simultaneously treated with the active principles and activated by a danger signal are still competent in their function of recognition of danger signals. To do this, we checked that the LDs/DDCs simultaneously treated with the active principles and stimulated with LPS and TNFα are capable of overexpressing the co-stimulation molecule CD86 after this stimulation.

The flow cytometry study proceeds in the following manner:

1/ The immature LCs/DDCs are generated as described in Example 1 and step 3.

2/ The cells are inoculated in P24-well plates, at a rate of 400 000 cells per cm², and in the culture medium described in Example 3. A mixture of LPS (at 50 μg/mL advantageously) and of TNFα (at 10 μg/mL advantageously) is added to the said culture medium. Simultaneously, the cells are treated with the active principles as described in Example 3.

3/ The cells that have been simultaneously treated with the active principles and stimulated with LPS and TNFα are analysed by flow cytometry, as described in Example 6, to quantify the residual expression of the molecule CD86 after “treatment+stimulation”.

4/ Table 9 shows the results of the analysis of the capacities for response to a danger signal of LDs/DDCs simultaneously treated with the selected active principles. The results are expressed in the following manner: the control not treated with the active agent and not stimulated with LPS+TNFα is referenced at 100% expression of the molecule CD86, and the tests are expressed as the residual expression of the molecule CD86 after “treatment”, after “treatment+stimulation” and “stimulation”. We also calculated the level of capacity for response to a danger signal, which is proportional to the residual expression of CD86 after treatment+stimulation.

TABLE 8
Study of the capacities for response to a danger signal of LCs/DDCs
simultaneously stimulated with LPS + 30TNFα and treated with the selected active
principles

| | RESIDUAL EXPRESSION | RESIDUAL EXPRESSION | RESIDUAL EXPRESSION | |
|---|---|---|---|---|
| ACTIVE | OF CD86 AFTER | OF CD86 AFTER | OF CD86 AFTER | RESPONSE |
| PRINCIPLES | TREATMENT | TREATMENT + STIMULATION | STIMULATION | CAPACITY |
| 8-Oxopseudopalmatine | 81% | 121% | | 0.85 |
| Securinine | 83% | 140% | | 0.98 |
| Foliosidine acetonide | 85% | 116% | | 0.81 |
| Phoradendron piperoides | 93% | 121% | | 0.85 |
| Cestrum latifolium | 51% | 86% | 143% | 0.4 |
| Spilanthes oleracea | 68% | 115% | | 0.80 |
| Maprounea guyanensis | 80% | 115% | | 0.80 |
| Dexamethasone | 50% | 75% | | 0.52 |

Conclusion:

LCs/DDCs simultaneously treated with the active principles and stimulated with TNFα/LPS have the capacity to respond to this danger signal, as demonstrated by the residual expression of the co-stimulation molecule CD86 after treatment+stimulation. The cells thus have a capacity for response to a danger signal close to that of their basal state.

### Example 9

### Study of the Capacities for Response to a Danger Signal of LCs/DDCs after Treatment with the Immunosuppressant Active Principles

As in Example 8, we checked that the LCs/DDCs are capable of responding to a danger signal, for example a bacterial infection, when they have been treated beforehand with the active principles and when they are no longer being treated. To do this, we checked that the LCs/DDCs treated with the said active principles, and then stimulated with LPS and TNFα (in the absence of the active principles), are capable of overexpressing the co-stimulation molecule CD86 after this stimulation.

The flow cytometry study proceeds in the following manner:

1/ The immature LCs/DDCs are generated as described in Example 1 and step 3.

2/ The cells are inoculated in P24-well plates, at a rate of 400 000 cells per cm², and in the culture medium described in Example 3. The treatment of the cells with the active principles is described in Example 3.

3/ The cells treated with the active principles are then recultured, without the active principles and in the culture medium described in Example 3, for a further 48 hours. A mixture of LPS (at 50 μg/mL advantageously) and of TNFα (at 10 μg/mL advantageously) is added to the said culture medium. The cells are then analysed by flow cytometry, as described in Example 6, to quantify the residual expression of the molecule CD86 after “pretreatment+stimulation”.

4/ Table 10 shows the results of the analysis of the capacities for response to a danger signal of LCs/DDCs treated beforehand with the selected active principles, and which are no longer being treated. The results are expressed in the following manner: the control not treated with the active agent and not stimulated with LPS and TNFα is referenced at 100% expression of the molecule CD86, and the tests are expressed as the residual expression of the molecule CD86 after “stimulation” and after “pretreatment+stimulation”. We also calculated the level of capacity for response to a danger signal, which is proportional to the residual expression of CD86 after pretreatment+stimulation.

TABLE 9
Study of thecapacities for response to a danger signal of LCs/DDCs pretreated
with the selectedactive principles and then stimulated with LPS + TNFα

| | RESIDUAL EXPRESSION OF CD86 | RESIDUAL EXPRESSION OF CD86 | |
|---|---|---|---|
| ACTIVE | AFTER | AFTER | RESPONSE |
| PRINCIPLES | STIMULATION | PRETREATMENT + STIMULATION | CAPACITY |
| 8-Oxopseudopalmatine | | 127% | 0.89 |
| Securinine | | 126% | 0.88 |
| Foliosidine acetonide | | 120% | 0.84 |
| Phoradendron piperoides | | 125% | 0.87 |
| Cestrum latifolium | 143% | 90% | 0.63 |
| Spilanthes oleracea | | 120% | 0.84 |
| Maprounea guyanensis | | 140% | 0.98 |
| Dexamethasone | | 130% | 0.91 |

Conclusion:

The LCs/DDCs pretreated with the active principles and then stimulated with TNFα/LPS have the capacity to respond to this danger signal, as demonstrated by the residual expression of the co-stimulation molecule CD86 after pretreatment+stimulation. The cells thus have a capacity for response to a danger signal close to that of their basal state.

### Example 10

### Use of the Products of the Invention in Cosmetic or Pharmaceutical Formulations of Oil-In-Water Emulsion Type

Formulation 10a:

| | | |
|---|---|---|
| A | Water | qs 100 |
| | Butylene Glycol | 2 |
| | Glycerol | 3 |
| | Sodium Dihydroxycetyl Phosphate, | 2 |
| | Isopropyl Hydroxycetyl Ether | |
| B | Glycol Stearate SE | 14 |
| | Triisononanoin | 5 |
| | Octyl Cocoate | 6 |
| C | Butylene Glycol, Methylparaben, | 2 |
| | Ethylparaben, Propylparaben, | |
| | pH adjusted to 5.5 | |
| D | Products of the invention of plant origin | 0.001%-10% |
| | Products of the invention of characterized origin | 10⁻⁷M − 1 M |

Formulation 10b:

| | | |
|---|---|---|
| A | Water | qs 100 |
| | Butylene Glycol | 2 |
| | Glycerin | 3 |
| | Polyacrylamide, Isoparaffin, Laureth-7 | 2.8 |
| B | Butylene Glycol, Methylparaben, | 2 |
| | Ethylparaben, Propylparaben; | |
| | Phenoxyethanol, Methylparaben, | 2 |
| | Propylparaben, Butylparaben, | |
| | Ethylparaben | |
| | Butylene Glycol | 0.5 |
| D | Products of the invention of plant origin | 0.001%-10% |
| | Products of the invention of characterized origin | 10-7M − 1 M |

Formulation 10c:

| | | |
|---|---|---|
| A | Carbomer | 0.50 |
| | Propylene Glycol | 3 |
| | Glycerin | 5 |
| | Water | qs 100 |
| B | Octyl Cocoate | 5 |
| | Bisabolol | 0.30 |
| | Dimethicone | 0.30 |
| C | Sodium Hydroxide | 1.60 |
| D | Phenoxyethanol, Methylparaben, | 0.50 |
| | Propylparaben, Butylparaben, | |
| | Ethylparaben | |
| E | Fragrance | 0.30 |
| F | Products of the invention of plant origin | 0.001%-10% |
| | Products of the invention of characterized origin | 10⁻⁷M − 1 M |

### Example 11

### Use of the Products of the Invention in a Formulation of Water-In-Oil Type

| | | |
|---|---|---|
| A | PEG 30-dipolyhydroxystearate | 3 |
| | Capric Triglycerides | 3 |
| | Cetearyl Octanoate | 4 |
| | Dibutyl Adipate | 3 |
| | Grape Seed Oil | 1.5 |
| | Jojoba Oil | 1.5 |
| | Phenoxyethanol, Methylparaben, | 0.5 |
| | Propylparaben, Butylparaben, | |
| | Ethylparaben | |
| B | Glycerin | 3 |
| | Butylene Glycol | 3 |
| | Magnesium Sulfate | 0.5 |
| | EDTA | 0.05 |
| | Water | qs 100 |
| C | Cyclomethicone | 1 |
| | Dimethicone | 1 |
| D | Fragrance | 0.3 |
| E | Products of the invention of plant origin | 0.001% - 10% |
| | Products of the invention of characterized origin | 10⁻⁷M − 1 M |

### Example 12

### Use of the Products of the Invention in a Formulation of Shampoo or Shower Gel Type

| | | |
|---|---|---|
| A | Xanthan Gum | 0.8 |
| | Water | qs 100 |
| B | Butylene Glycol, Methylparaben, | 0.5 |
| | Ethylparaben, Propylparaben | |
| | Phenoxyethanol, Methylparaben, | 0.5 |
| | Propylparaben, Butylparaben, | |
| | Ethylparaben | |
| C | Citric acid | 0.8 |
| D | Sodium Laureth Sulfate | 40.0 |
| E | Products of the invention of plant origin | 0.001%-10% |
| | Products of the invention of characterized origin | 10⁻⁷M − 1 M |

### Example 13

### Use of the Products of the Invention in a Formulation of Lipstick Type and other Anhydrous Products

| | | |
|---|---|---|
| A | Mineral Wax | 17.0 |
| | Isostearyl lsostearate | 31.5 |
| | Propylene Glycol Dipelargonate | 2.6 |
| | Propylene Glycol Isostearate | 1.7 |
| | PEG 8 Beeswax | 3.0 |
| | Hydrogenated Palm Kernel Oil Glycerides, | 3.4 |
| | Hydrogenated Palm Glycerides | |
| | Lanolin Oil | 3.4 |
| | Sesame Oil | 1.7 |
| | Cetyl Lactate | 1.7 |
| | Mineral Oil, Lanolin Alcohol | 3.0 |
| B | Castor Oil | qs 100 |
| | Titanium Dioxide | 3.9 |
| | Cl 15850:1 | 0.616 |
| | Cl 45410:1 | 0.256 |
| | C191401 | 0.048 |
| | C177491 | 2.048 |
| C | Products of the invention of plant origin | 0.001%-10% |
| | Products of the invention of characterized origin | 10⁻⁷ M − 1 M |

### Example 14

### Use of the Products of the Invention in an Aqueous Gel Formulation (Eye Contour, Slimming, etc.)

| | | |
|---|---|---|
| A | Water | qs 100 |
| | Carbomer | 0.5 |
| | Butylene Glycol | 15 |
| | Phenoxyethanol, Methylparaben, Propylparaben, | 0.5 |
| | Butylparaben, Ethylparaben | |
| B | Products of the invention of plant origin | 0.001%-10% |
| | Products of the invention of characterized origin | 10⁻⁷M − 1 M |

### Example 15

### Use of the Products of the Invention in a Formulation of Triple Emulsion Type

Primary emulsion W1/O:

| | | |
|---|---|---|
| A | PEG 30-dipolyhydroxystearate | 4 |
| | Capric Triglycerides | 7.5 |
| | lsohexadecane | 15 |
| | PPG-15 Stearyl ether | 7.5 |
| B | Water | 65.3 |
| C | Phenoxyethanol, Methylparaben, | 0.7 |
| | Propylparaben, Butylparaben, | |
| | Ethylparaben | |

Secondary emulsion W1/O/W2:

| | | |
|---|---|---|
| A | Primary emulsion | 60 |
| B | Poloxamer 407 | 2 |
| | Phenoxyethanol, Methylparaben, | 0.3 |
| | Propylparaben, 2-bromo- | |
| | 2-nitropropane-1,3-diol | |
| | Water | qs 100 |
| C | Carbomer | 15 |
| D | Triethanolamine | pH 6.0-6.5 |
| E | Products of the invention of plant origin | 0.001%-10% |
| | Products of the invention of characterized origin | 10⁻⁷M − 1 M |

### Example 16

### Preparation of Pharmaceutical Formulations Containing the Product of the Invention

Formulation 16a: Preparation of Tablets

| | | |
|---|---|---|
| A | Excipients | In g per tablet |
| | Lactose | 0.359 |
| | Saccharose | 0.240 |
| B | Products of the invention of plant origin | 0.001%-10% |
| | Products of the invention of characterized origin | 10⁻⁷M − 1 M |

Formulation 16b: Preparation of an Ointment

| | | |
|---|---|---|
| A | Excipients | |
| | Low-density polyethylene | 5.5 |
| | Liquid paraffin | qs 100 |
| B | Products of the invention of plant origin | 0.001%-10% |
| | Products of the invention of characterized origin | 10⁻⁷M − 1 M |

Formulation 16c: Preparation of an Injectable Formula

| | | |
|---|---|---|
| A | Excipient | |
| | Isotonic saline solution | 5 ml |
| B | Products of the invention of plant origin | 0.001%-10% |
| | Products of the invention of characterized origin | 10⁻⁷M − 1M |

1. Use of at least one active principle chosen from a plant extract of Cestrum latifolium, a plant extract of Phoradendron piperoides, a plant extract of Spilanthes oleracea, a plant extract of Maprounea guyanensis, securinine, foliosidine acetonide, 8-oxopseudopalmatine, or any combination thereof, as an active agent for increasing the activation threshold of the immune cells of an individual, in a cosmetic composition, or for the preparation of a pharmaceutical composition, and in particular of a dermatological composition, preferably intended for increasing the activation threshold of the immune response of an individual.

2. Use of at least one active principle according to claim 1, for reducing and/or preventing the cutaneous and/or mucosal immune reaction.

3. Use of at least one active principle according to claim 1, for reducing and/or inhibiting the activity of at least one type of immune cell, in particular dendritic cells.

4. Use of at least one active principle according to claim 1, for reducing the activity of at least epidermal Langerhans cells (LC) and/or dermal dendritic cells (DDC).

5. Use of at least one active principle according to claim 1, in combination with a calming agent.

6. Use of at least one active principle according to claim 1, characterized in that the amount of the plant extract is between 0.001% and 10% by weight and preferentially between 0.01% and 5% by weight of the composition.

7. Use of at least one active principle according to any claim 1, characterized in that the amount of securinine, foliosidine acetonide, and/or 8-oxopseudopalmatine is between 1×10⁻⁷% and 1% by weight and advantageously between 1×10⁻⁵% and 1×10⁻¹% by weight relative to the total weight of the composition.

8. Use of at least one active principle according to claim 1, characterized in that the active principle is a plant extract of Cestrum latifolium and/or securinine.

9. Use of at least one active principle according to claim 1, for reducing in the skin the expression of at least one type of marker chosen from (i) CD86, expressed at a basal level by the immature dendritic cells; (ii) CD40, CD54, CD80, HLA-DR, CCR7 or CD86 expressed by activated dendritic cells; and (iii) CD40, CD54, CD80, HLA-DR, CCR7, CD86, CD83 or DC-LAMP expressed by activated and mature dendritic cells.

10. Use of at least one active principle according to claim 1, in a cosmetic method for caring for and/or treating sensitive, reactive and/or hyperreactive skin.

11. Use of at least one active principle chosen from a plant extract of Cestrum latifolium, a plant extract of Phoradendron piperoides, a plant extract of Spilanthes oleracea, a plant extract of Maprounea guyanensis, securinine, foliosidine acetonide, 8-oxopseudopalmatine, or any combination thereof, in a cosmetic care and/or treatment method for reducing and/or preventing unaesthetic and/or uncomfortable manifestations, especially stinging, sensations of burning, sensations of itching, tautness, visible squamae and thickening of the skin.

12. Use of at least one active principle according to claim 1, for the preparation of a pharmaceutical composition, and especially a dermatological composition, for preventing and/or treating an irritation or cutaneous and/or mucosal allergic reaction, especially a contact reaction, a delayed contact hypersensitivity, a contact eczema, an urticaria, especially contact urticaria, an irritative or allergic contact dermatitis, and/or an atopic dermatitis.

13. Use of at least one active principle according to claim 12, for the preparation of a pharmaceutical composition also comprising an anti-inflammatory agent.

14. Cosmetic composition comprising an active principle chosen from a plant extract of Cestrum latifolium, a plant extract of Phoradendron piperoides, a plant extract of Spilanthes oleracea at a concentration of between 0.001% and 10% by weight, securinine at a concentration of from 1×10⁻⁷% to 1%, foliosidine acetonide, 8-oxopseudopalmatine, and any combination thereof, in a cosmetic vehicle suitable for topical application.

15-16. (canceled)


SEQUENCE LISTING (4 sequences)

SEQ ID NO: 1 - DNA, 20 bp, artificial sequence
  actin sense primer
gtggggcgccccaggcacca

SEQ ID NO: 2 - DNA, 24 bp, artificial sequence
  actin antisense primer
ctccttaatgtcacgcacgatttc

SEQ ID NO: 3 - DNA, 22 bp, artificial sequence
  CD86 sense primer
ctctttgtgatggccttcctgc

SEQ ID NO: 4 - DNA, 22 bp, artificial sequence
  CD86 antisense primer
cctgtgggctttttgtgatgga